(19) United States
(12) Patent Application Publication
(10) Pub. No.: US 2011/0014671 A1
(43) Pub. Date: Jan. 20, 2011

(54) METHOD FOR OBTAINING BIOETHANOL FROM SORGHUM GRAIN (SORGHUM BICOLOR L. MOENCH), COMPRISING STEPS INVOLVING DECORTICATION AND HYDROLYSIS WITH PROTEASES

(75) Inventors: Sergio Roman Othon Serna Saldivar, Garza Garcia (MX); Esther Perez Carrillo, San Nicolas de los Garza (MX); Mario Moises Alvarez, Monterrey (MX)

Correspondence Address: BAKER & MCKENZIE LLP, Pennzoil Place, South Tower, 711 Louisiana, Suite 3400, Houston, TX, 77002, US

(73) Assignee: INSTITUTO TECNOLOGICO Y DE ESTUDIOS SUPERIORES DE, MONTERREY (MX)

(21) Appl. No.: 12/808,172

(22) PCT Filed: Oct. 24, 2008
(86) PCT No.: PCT/MX08/00144

§ 371 Date: Sep. 14, 2010

(30) Foreign Application Priority Data
Dec. 14, 2007 (MX) MX/A/2007/016073

(51) Int. Cl. C12P 7/06

(52) U.S. Cl. 435/161

(57) ABSTRACT

The invention relates to an improved method for obtaining bioethanol from sorghum grain (Sorghum bicolor L. Moench), comprising steps involving decortication and hydrolysis with proteases prior to liquefaction. The decortication is performed mechanically and produces a by-product rich in pericarp or bran and stable sorghum grain. The decorticated grain produces greater quantities of bioethanol compared to the whole grain. The hydrolysis with protease, performed before and during the first part of the liquefaction step, enables the hydrolysis of the protein matrix covering the sorghum starch granules and provides a higher rate of hydrolysis of starch to dextrins and glucose after the liquefaction and saccharification steps respectively. The two novel steps of the method significantly reduce the biocatalysis and fermentation times such that ethanol can be produced in less time and using fewer resources.

### OBJECT OF THE INVENTION

The object of the invention concerns an improved process that allows a greater yield of bioethanol to be obtained from sorghum grain (Sorghum bicolor L. Moench), and commercially improved by-products such as decortication fiber and spent grain from distilleries obtained from sorghum grain.

### BACKGROUND

Bioethanol is a fuel that serves as an alternative to gasoline in the process of internal combustion. The resurgence of the use of bioethanol as a fuel is due to two main reasons: reduction of the country's dependence on oil supplies or deposits and greater ecological awareness that has caused us to seek a lower impact on the environment. There are many energy sources and technologies that may be considered to reduce or replace the dependence on petroleum and its derivatives. However, technological alternatives are reduced if you take into account that (Sanderson, 2007):
Supply lines are needed that are unlikely to suffer interruptions and negative financial effects due to international problems, such as instability in the price of petroleum and natural disasters.
It must stimulate the rural economy as much as possible, increasing opportunities in the marketing field.
It must promote protection of the environment, through the reduction in emissions of contaminants and a reduction in the destruction of habitat and natural resources.

Taking the above into account, bioethanol has been promoted as a solution for a group of complex problems related to energy and the environment. Compared to purchasing fossil fuels, bioethanol has great advantages because it is a renewable resource, paving the way for clean combustion and production of a lower amount of greenhouse gases (Altintas et al. 2002). Based on the premise that bioethanol may contribute to a cleaner environment, and with implementation of laws to protect the environment in different parts of the world, governments have established strategies to propel its production and use as a fuel (Ccopa-Rivera et al. 2006).

For Brazil, the largest source for bioethanol production has been and still is: sugar cane (Saccharum officinarum). This country was one of the first to boost the production and use of bioethanol during the 1970s and 80s (Ccopa-Rivera et al. 2006). In the case of the United States, the commercial production of ethanol as a biofuel has had an unprecedented growth and an increase in ethanol production for use as a fuel is expected (Srinivasan et al. 2006; Sanderson, 2007). Approximately 95% of this production is from corn (Zea mays L), mainly processed by dry grinding and the rest from several cereals among which sorghum is found (Wu et al. 2007).

One of the main arguments against the use of bioethanol is its financial competitiveness against fossil fuels. However, through Brazil's experience it has been demonstrated that production scale, and technological advances may pave the way for competitiveness in the production of bioethanol reducing the gap between bioethanol and conventional fossil fuels (Goldemberg et al. 2004). Consequently, there is great interest in studying all stages involved in the production of bioethanol for the purpose of reducing production costs (Ccopa-Rivera et al. 2006). Different raw materials have been considered (Jones et al. 1994), variations in grinding conditions, improvement in the production process of fermentable sugars, fermentation at high concentrations of sugars and nitrogen (Jones et al. 1994), continuous yeast culture with recirculation (Brandberg et al. 2007) and recombinant microorganism use (Altintas et al. 2002), among many other alternatives.

The use of starchy cereals as a substrate for alcohol fermentation with yeast (Saccharomyces cerevisiae) is a well established, recognized, and mature technology (Pimientel y Patzek, 2005). Starch, the main storage polysacharride, that represents up to 70% of the weight of corn, is a high yield source for glucose (Bello-Perez et al. 2002; Nigam and Singh, 1995). In comparison with other fermentable sugar sources, cereals are advantageous because it is possible to store them for prolonged periods and they have low transportation and pre-treatment costs (Abouizied and Reddy, 1986).

To produce bioethanol from cereals, starch must by hydrolyzed to fermentable sugars, mainly glucose (Thomas and Ingledew, 1990). The stages that traditionally are performed are: gelatinization of starch granules, liquefaction with generally thermo-resistant alpha-amylase and saccharification with amyloglucosidase. These two enzymes (alpha-amylase and amyloglucosidase) convert practically all the starch present in the grain into fermentable sugars, mainly glucose. Afterwards using fermentation, the fermentable sugars are converted to bioethanol. Due to the strong demand for bioethanol in the world, the demand for enzymes and yeasts has increased significantly. The application of biocatalysis technology for the production of ethanol may be classified in two areas, alcoholic drinks and ethanol fuel. The technology is generally the same, however, some of the conditions and substrates are different (Sears, 1995). In the case of bioethanol, a reduction in energy requirements is being sought to make it more competitive with fossil fuels (Wang et al. 2007a). Yield is the most important consideration for the production of bioethanol from corn, and there is a renewed emphasis on the design of cooking processes that permit a greater yield (Maisch, 2003).

The objective of the saccharification process is the production of a syrup with the greatest amount of glucose possible. The reason high glucose levels (>94%) are not reached is generally attributed to the reversion reaction catalyzed by the amyloglucosidase. An additional factor has also be recognized for not obtaining high yields is the formation of maltulose (4-α-D-glucopyranosyl-beta-D-fructose). Once maltulose forms, the final yield of glucose is reduced because amyloglucosidase does not act between the glucose and the fructose (Guzman-Maldonado y Paredes-Lopez, 1995).

In addition to the presence of fermentable sugars, which are the carbon source for yeast, a source of nitrogen is required. Even when a cereal is rich in proteins, yeast cannot use this complex material unless it is first hydrolyzed to amino acids, dipeptides, or at least to tripeptides (Thomas y Ingledew, 1990). Other sources of non-protein nitrogens may be used such as ammonia and urea. These sources are added to support the growth of yeast and to avoid defective fermentations. The amount and type of nitrogen source not only affects the growth but also has an effect on the ethanol yield and the type of associated by-products (Wu et al. 2006b).

One of the principal factors in the cost of the bioethanol production process is the raw material (Brandberg et al. 2007). In the selection of the raw materials, mainly market cost should be taken into consideration and in the case of cereals, starch is the most bioavailable. This is because different types of cereals affect the speed of conversion and the final ethanol yield in an important way (Wu et al. 2006a). In general terms, the cost of the raw materials affect 70% of the ethanol production cost produced from starchy cereals.

Biorefineries that process cereals generally have a plant design based on dry grinding instead of wet grinding (refined starch) because this requires a lower initial and equipment investment (Srinivasan et al. 2006). In the case of corn, the grain is ground and mixed with water to form a suspension which is heated, dextrinized, sacarified, and fermented to produce bioethanol. The non-fermentable materials of corn (germ, fiber, and protein) are recovered at the end of the process as spent distillery grain or GGD. The net cost of corn is high in the dry grinding process because of the low value of the GGD (Singh et al. 2005). With the purpose of making this process more profitable, work has been developed to increase the ethanol yield and the efficiency of the conversion.

Thomas et al. (2001) studied the effect of lactobacillus on the growth of semi-continuous fermenting yeast using corn as a raw material.

Pimientel and Patzek (2005) carried out a comparative study taking into account the investment in the field and during the ethanol production process from corn, the principal investments being in machinery, irrigation, and herbicides.

Singh et al. (2005) proposes the technology of dry enzyme fractionated grinding for the purpose of improving the quality of the spent distillery grain, increasing its market value and at the same time increasing the ethanol yield. In this process, the corn is soaked in water for a short period followed by rough grinding with water (in addition to the soaking water) to selectively remove the pericarp and starch free germ. The resulting endosperm is finely ground and the remaining suspension dextrinized, sacarified, and fermented to produce bioethanol. The protein content of the GGD from the dry enzyme grinding was 58% and the acid detergent fiber was 2%, in comparison with 28% protein and 11% acid detergent fiber in the traditional GGD.

Wang et al. (2005), concluded that when using a dry grinding process with proteolytic enzymes in corn, the concentration of fermentable substrates are increased producing the advantage of a lower cost in the purification stage. It should be mentioned that these authors added the enzymes in the saccharification/fermentation stage.

Singh et al. (2006) performed work related to the endogenous α-amylases in corn to reduce the cost involved in liquefaction.

In the case of Mexico, the most recent information on the total energy consumption in the country is from the year 2005 and according to the 2005 National Energy Report (SENER, 2007), 42.5% was in transportation. At the same time, 90% of the demand in transportation is gasoline, which means that 38.58% of the total energy consumption in Mexico in 2005 was used in trucking which involves gasoline motors. This represents the greatest energy demand. The scenario is not very encouraging in the short term because of two central facts: after 2006 there has been a gradual reduction in oil production at Canterell, a source that provides 61% of the national production (PEMEX, 2007), and the problem has worsened because vehicle fleets are on the increase every day and consequently so is the demand for fuel. Being aware of this, on Apr. 26, 2007, the Senate of the Republic approved the “Bioenergy Development Law”.

It is important to note that in the selection of raw materials to produce bioethanol one must take the availability of these into account and it should be of low cost. In Mexico, sugar cane is “politically impossible” due to the “Cane Sugar Law” and the high price of sugar cane on the market (40 dollars a ton). Also, an increase in demand is expected and therefore an increase in the price of sugar cane since the production of High Fructose Corn Syrup will be reduced by channeling the majority of corn production to the production of bioethanol (Quadri, 2007).

As far as corn is concerned, in Mexico it should not be considered as an alternative for the production of bioethanol fuel, since it makes up a basic part of the diet of the poorest portion of the population. Moreover, sorghum (Sorghum bicolor) is a cereal characterized by a chemical composition that is very similar to that of corn, but with fewer field and irrigation requirements for its cultivation (Hubbard et al. 1950).

In the western world, sorghum has long been used as a crop for cattle feed, but the large amounts of same that were processed during the Second World War by the distilling and grinding industry shown the potential of this grain as raw material for fermentation processes especially in zones where growing corn is difficult, such as in arid or semiarid zones (Hubbard et al. 1950; Lázaro and Favier, 2000). Mexico is the fourth largest producer of sorghum in the world with the great majority being used for poultry and cattle feed. The chemical composition of sorghum is practically identical to that of corn. Generally speaking, it contains a little more protein but a lower concentration of oil. Interestingly, the sorghum grain contains approximately the same amount of starch compared to corn kernels, which makes it attractive as raw material for the production of bioethanol. In regards to the use of sorghum as raw material for industrial processing, the following has been observed.

In a conventional process to obtain ethanol, where corn kernels are substituted by sorghum grain, the recovery of starch from sorghum grain is not as complete as when corn kernels are used. Some of the factors responsible for this situation are: (a) this starch is lost in the fraction of germ and fiber because starch may be found in the pericarp of the grain and some of these peripheral cells are not cracked during grinding, and (b) starch is also found covered by highly interwoven proteins (Yang y Seib, 1996).

Some peripheral endosperm cells do not open during the wet grinding process and the high level of interwoven kafirins or prolamines associated with the starch granules makes fractioning of the sorghum more difficult (Higiro et al. 2003).

Abrasive decortications techniques have traditionally been used to obtain flours and grits refined from sorghum in Africa and Asia and the by-products rich in fiber, oil, protein, and energy derived from the decortications may be used and channeled into feed for cattle, mainly ruminants (Rooney et al. 1972).

Refined grits with a higher starch content have also been used as adjuncts in the beer industry given that the malt enzymes transform them into dextrin and fermentable carbohydrates (Higiro et al. 2003).

Certain sorghum processing methods have been observed to increase the digestibility of its proteins. Hamaker et al. (1987), observed that “in vitro” digestibility of sorghum proteins is negatively affected by the cooking process, while the use of reducing agents (mercaptoethanol) improve the digestibility thereof. These authors concluded that thermoplastic extrusion as well as fermentation increased the digestibility of sorghum when used in baby food.

One of the most important steps in wet grinding to obtain starch, is the stage in which it is soaked in a weak sulfur dioxide solution. This is due to the time involved (48 hours) and because it is a process performed in batches. Several reports have been published the objective of which is to improve hydrolysis of the proteins in sorghum grain to release the starch rapidly and effectively.

Yang and Seib (1996), tried to optimize the soaking process increasing the temperature to reduce the soaking time, but they did not obtain favorable results. They arrived at the conclusion that half of the starch is found in association with interwoven proteins.

The results obtained by these authors indicate that the initial bottleneck in isolating starch from the grains of sorghum was because the soaking liquid becomes diffused within the grain. The second barrier would probably be the high level of interwoven sorghum proteins. In addition, Johnston and Singh (2001), observed that the use of proteases reduces the soaking time and the requirements for SO2 in the case of corn, but they observed no advantage at all regarding the use of cell wall enzymes, either alone or combined with proteases.

Moheno-Pérez et al. (1997), Wang et al. (2000) and Serna-Saldivar and Mezo-Villanueva (2003), have carried out work also focused on the soaking stage, but in terms of the use of cell wall enzymes, with favorable results regarding starch yield.

Mezzo-Villanueva and Serna Saldivar (2004), carried out work concerning the use of proteases during the sulfur dioxide soaking stage obtaining favorable results in starch yield.

Moheno-Perez (1994) observed that when cell wall degrading enzymes are added to the soaking solution (a single stage), the starch yield obtained from whole sorghum grain was not improved. In a prior study, Serna-Saldivar and Mezo-Villanueva (2003), observed that the use of cell wall degrading enzymes significantly increased the yield and recovery of starch, when they are added in the stage prior to soaking the pre-grinded grain at a concentration of 120 FBG/100 mL of grain.

Perez-Carrillo and Serna-Saldivar (2006) observed that using protease in cracked grain after soaking, either alone or in combination with cell wall degrading enzymes, produced results of greater starch yield through wet grinding. This brought them to the conclusion that that the greater effect on the availability of starch is due to the presence of proteins interacting with the starch granules.

It has also been demonstrated that starch yield may be substantially increased by submitting cracked corn kernels to an enzymatic attack in the soaking stage. However, because the wet grinding process has been practically perfected without the addition of enzymes and because starch granules are not highly interwoven with the protein matrix, in practice, these catalysts are not used. On the other hand, the use of enzymes in wet grinding or starch refining from sorghum is more effective because it has a protein matrix that is more interwoven and difficult to hydrolyze.

In the case of breweries, it is well known that unmalted sorghum contains practically no endogenous enzymes which consequently give musts with a low concentration of fermentable sugars. This makes it necessary to use exogenous enzymes and to take into account different technological aspects during maceration such as: the capacity of the cooker, the energy expense, the use of endogenous enzymes, and the optimal parameters thereof. An improper maceration stage will cause insufficient proteolysis, incomplete gelatinization of the starch, which results in a low grade saccharification and musts with filtration problems (Goode et al. 2003). Interestingly, the same authors observed that the use of protease in the production of musts from unmalted sorghum increased the level of protein hydrolysis and nitrogen solubilization.

The conventional sorghum processing technology is very far from optimal in comparison with other cereals of equal relevance such as corn, wheat (Triticum sp) and rice (Oryza sativa) (Lazaro and Favier, 2000). One of the problems involved in the correct use of sorghum is ineffective decortication (Suroso et al. 2000). The amounts of certain anti-nutrients may be reduced by decortications since it may selectively remove the pericarp and the chime (Lestienne et al. 2006). Yetneberk et al. (2005) observed that when sorghum undergoes decortication the quality of the fermented food product is improved. Corredor et al. (2006) studied the effect of using only 10 and 20% sorghum decortication on the production of bioethanol and the composition of distillery grains, concluding that 10% decortications had a greater positive effect than 20% decortications in regards to biofuel yield. This last work shows the possible use of decortication, not only in the production of food, but as a prior stage in the process of fuel grade alcohol production. Lantero et al. (1993) presented a patent in which they propose the use of a protease in the simultaneous saccharification-fermentation stage. The use of the proteolytic enzyme had a positive effect on the production of ethanol and nitrogen or amino acids available to the yeast. However, the technology proposed has the disadvantage of not allowing the release of dextrins trapped by the proteins, plus the proteolytic enzyme may significantly lower the activity of the glucoamylase or amyloglucosidase.

In the patent application, which is the object of this invention, an improved process is referenced for the production of bioethanol using as raw material, sorghum grain (Sorghum bicolor 1. Moench); and the products obtained are bioethanol, decortication fiber, and GDD obtained from the sorghum grain.

### BRIEF DESCRIPTION OF THE FIGURES

FIG. 1. Flow chart of the improved process for obtaining bioethanol from sorghum grain.

FIG. 2. Graph of the percentage of fiber withdrawn expressed based on the initial weight of decorticated white sorghum with respect to the time of decortication.

FIG. 3. Graph of the profile of the concentration of reducing sugars in the hydrolyzed flour suspensions with heat-stable α-amylase in the conventional process.

FIG. 4. Graph of the profile of the concentration of reducing sugars in the hydrolyzed flour suspensions with heat-stable α-amylase in the conventional process.

FIG. 5. Graph of the profile of the concentration of α-ANL during the saccharification stage with heat-stable α-amylase in the conventional process.

FIG. 6. Graph of the profile of concentration of α-ANL during the stage of saccharification with heat-stable α-amylase in the process proposed here that includes hydrolysis with protease.

FIG. 7. Graph of the profile of glucose concentration in the dextrin suspension in the conventional process.

FIG. 8. Graph of the profile of glucose concentration in the dextrin suspension in the process here proposed that includes protease hydrolysis.

FIG. 9. Graph of the profile of glucose concentration in the sweet must during the fermentation stage in the conventional process.

FIG. 10. Graph of the profile of glucose concentration in the sweet must during the fermentation stage in the process here proposed that includes protease hydrolysis.

FIG. 11. Graph of the profile of the α-ANL concentration in the sweet must during the fermentation stage in the conventional process.

FIG. 12. Graph of the profile of the α-ANL concentration in the sweet must during the fermentation stage in the process here proposed that includes protease hydrolysis.

FIG. 13. Graph of the profile of ethanol generation during the fermentation stage of sweet musts in the conventional treatment.

FIG. 14. Graph of the profile of ethanol generation during the fermentation stage of sweet musts in the process here proposed that includes protease hydrolysis.

### DETAILED DESCRIPTION OF THE INVENTION

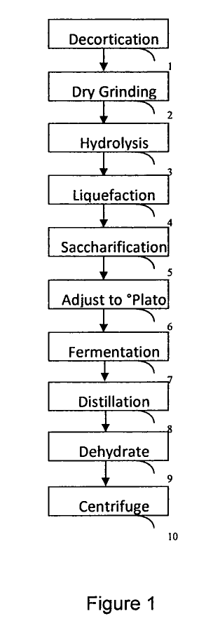

This invention addresses an improved process for the production of bioethanol using sorghum grain as the raw material (Sorghum bicolor 1. Moench). The general process is shown in FIG. 1 and includes stages of:

### a) Decorticate the Sorghum Grain (1)

This stage is carried out to remove the outer layers of the sorghum grain, to expose the endosperm that contains the starch, and to make it vulnerable and to pave the way for its conversion to bioethanol in the later stages.

Decortication is optimally carried out until 7-10% by weight of the whole grain is removed by mechanical means, such as for example, equipment provided with carborundum discs, (Mutana Machine, Saskatoon). Additionally, in this stage more than 60% of phenol compounds are removed from the whole sorghum grain, and the removal of these phenol compounds, concentrated in the decortication fiber which in a conventional process for bioethanol production, is not obtained. The resulting products from the decortication stage are: decorticated sorghum grain and decortication fiber (which includes: the pericarp, or bran, and part of the germ). Preferably, the sorghum grain is decorticated from 5 to 30 minutes until between 7 and 10% of the original weight of the grain is removed that, during the stages of the process which is the motive of this invention, translates mainly into energy savings and high efficiency; because the amount of fermentable material in the decorticated sorghum grain that enters the process have greater bioavailability and greater proportion; and the concentration of non-fermentable material (the decortication fiber) that enters the process and that involves the greatest energy consumption; and that upon being separated, this decortication fiber may be commercially marketed.

During this stage, some grains of sorghum are cracked, and to reduce the loss of cracked grain, the products resulting from the decortication are treated with a medium capable of removing only the decortication fiber (for example, an air aspirator or fanning mill) and separate it selectively from the decorticated grain.

By removing the decortication fiber from the decorticated grain (which is the main product of this stage), its concentration of phenol compounds is reduced and it is converted into a substrate with greater bioavailability for: amylolytic enzymes used in later liquefaction and liquefaction stages and for the yeasts in the fermentation stage.

The decortication fiber obtained has a moisture content of between 9 to 15%, and may be used mainly for cattle feed or as raw material in the preparation of food for human consumption, because it is characterized by being rich in proteins, ethereal extract or oil, low moisture, and phenol compounds; which confers storage stability at room temperature to it, and the consequent reduction in transportation costs; extending its shelf life, since the phenol compounds act as protective agents against illnesses caused by insects and phytopathogens and as antioxidants that prevents the oil from becoming rancid which obviously lowers the palatability of the cattle feed.

### b) Dry Grind the Decorticated Sorghum Grain (2)

The decorticated sorghum grain is crushed or ground preferably using a Arthur Thomas Co. (Philadelphia, Pa.) hammer mill provided with a mesh having 2 mm holes and the product obtained is sorghum flour; the resulting sorghum flour had a particle size distribution or granulometric profile with 68% of the particles having a size of 420 μm, 11.5% between 250 and 420 μm, 7.7% between 149 and 250 μm and 11.1 smaller than 149 μm. The sorghum flour and the decortication fiber were chemically described, and the results obtained are presented in Table 1, from these results we may stress that the greatest concentration of starch is in the sorghum flour and it has a lower concentration of phenol compounds.

TABLE 1
Chemical description and profile of total phenols in
the decorticated sorghum flour (Sorghum bicolor L. Moench)
and decortication sorghum fiber flour¹.

| | Decorticated | Decortication |
|---|---|---|
| Parameter | Sorghum Flour | Fiber |
| Moisture | 11.15 ± 0.05 | 8.88 ± 0.04 |
| Ash | 1.36 ± 0.03 | 3.96 ± 0.05 |
| Protein | 10.90 ± 0.62 | 18.07 ± 0.20 |
| Ethereal Extract | 0.66 ± 0.05 | 10.43 ± 0.44 |
| Raw Fiber | 1.78 ± 0.14 | 11.08 ± 0.24 |
| ELN | 83.80 ± 0.84 | 76.26 ± 0.76 |
| Starch | 75.80 ± 0.22 | 19.80 ± 0.41 |
| Total Phenol | 0.45 ± 0.01 | 10.92 ± 0.72 |
| (equivalent to gallic | | |
| acid mg/g) | | |

¹The reported values correspond to the average of three repetitions and are reported as a percentage of the dry base, with the exception of the total phenols.
ELN = Free nitrogen extract which gives an indication of the amount of starch and other sugars.

c) Hydrolyze with Protease (3)

From the sorghum flour obtained by dry grinding, it is diluted in water to obtain a flour water suspension that is submitted to a temperature ramp as indicated below:
Prepare a 30:70 suspension of flour:water.
Add between 0.2 and 0.4% Ca(OH)2 with respect to the content of the sorghum flour to ensure good activity from the calcium dependent enzymes.
Adjust the flour:water suspension to a pH of 6.5 using HCl 0.1 N. Optionally you can work with a pH between 4.5 to 7.
Add 0.5 mL of at least one proteolytic enzyme.
Heat in a bain-marie and stir the flour:water suspension, seeing that it reaches a temperature of 60° C. within 15 minutes. It is important to note that the temperature condition is not the temperature recommended as optimal for the enzyme supplier, however it is the optimal temperature range for this stage, that in a conventional process the temperature increase is not used as we will use it in the process here proposed, that uses it to include hydrolysis with at least one protease.
   According to the manufacturer, the optimal conditions for the metalloprotease mixture used are:
Temperature within the range of 45 to 55° C. and it becomes denatured or inactive at temperatures higher than 70° C.
pH in the range of 5.5 to 7.5, may use one or a combination of proteolytic enzymes with characteristics similar to this one.

### d) Liquefy the Hydrolyzed Flour:Water Suspension (4)

From the hydrolyzed flour:water suspension obtained in the previous stage, a suspension of dextrins is obtained, with the protocol indicated below:
Add 0.2 mL of a heat-resistant alpha-amylase to the hydrolyzed flour:water suspension. The activity declared by the supplier for this enzyme is 240 KNUs/g. Works optimally at 90° C. and at a pH of 7.
Increase the temperature gradually for 55 minutes until the maximum temperature of 83 to 95° C. is reached. (It is important to note that this temperature is not the optimal temperature for the activity of the enzyme recommended by the supplier, but it is the temperature recommended to carry out this stage.)
Allow reaction to take place for 2 hours at 83° C.-90° C. while shaking at 50-150 rpm to ensure contact between the enzyme and the substrate.
Reduce the temperature to a range between 55 and 60° C. within the least amount of time possible.
Adjust the dextrin suspension to a pH of 5.5 using HCl 0.1 N.
   According to the manufacturer, the optimal conditions for the heat-resistant alpha-amylase are:
Temperature within the range of 85° C.-95° C. and pH in the range of 5.5-7.5. However, this heat-stable alpha-amylase enzyme, may be substituted by any commercial enzyme having similar characteristics.

In this stage of liquefaction, the starch of the hydrolyzed flour:water suspension, first presents gelatinization, a phenomenon that may be affected by the characteristics of the starch granule. Gelatinization is necessary to increase the efficiency of the heat-resistant alpha-amylase enzyme because the starch granules crack exposing the amylose and amylopectin molecules that are most vulnerable to the heat-resistant alpha-amylase.

### e) Saccharification of the Dextrin Suspension (5)

Convert the dextrin suspension obtained in the liquefaction stage into a concentrated sweet must suspension. In order to carry out the conversion, the amyloglucosidase enzyme is added to the dextrin suspension in the previous stage (liquefaction), in a ratio of enzyme:substrate 1:100 (p/p). Incubate the dextrin suspension at a temperature ranging from 55 to 60° C. and at a pH of 5.5 while shaking at between 60 and 120 rpm, for between 14 and 16 hours to obtain a concentrated sweet must with insoluble material.

According to the supplier, the amyloglucosidase enzyme works optimally at a pH ranging between 4.5 and 5.5 and at an optimal temperature of 55° C. to 60° C. and consists of an enzymatic complex comprising principally amyloglucosidase or glucoamylase and enzymes that debranch the starch chains called pullulanases.

### f) Adjust to Degrees Plato for the Concentrated Sweet Musts (6)

Once the saccharification stage is finished, the concentrated sweet musts are cooled between 20 and 25° C. and they are adjusted to a certain sugar concentration. For the adjustment, the concentration of soluble solids should be measured as ° Plato using a refractometer. If the soluble solid concentration in the concentrated sweet musts is at a value above 11-15 ° Plato, water should preferably be added to dilute it and to obtain a refractometer reading of between 11-15 ° Plato and when the concentrated sweet musts are diluted it hereinafter will be called standardized sweet must.

### g) Ferment the Standardized Sweet Must (7)

In this stage the standardized sweet musts are preferably inoculated with a yeast to obtain bioethanol, which is the product of interest in the process here proposed. In order to do this, it is necessary:
To adjust the standardized sweet musts preferably to a pH of 4.5 to 5.5 and at an optimal temperature of between 32 and 36° C.
Inoculate the standardized sweet musts with a yeast, (genetically modified osmotolerant organisms), or another microorganism capable of converting glucose into ethanol. Examples of yeasts may be Pichia pastoris sp or Saccharomyces cereviseae. Saccharomyces cereviseae ATCC 24858 are preferably used in this process in a 1:100 ratio. This means that 1 mL of inoculum is added for every 100 mL of sweet must to begin fermentation with a concentration of 15×106 cells/900 mL of standardized sweet must.
   Incubate the inoculated sweet musts preferably at 30° C. with shaking at 125 rpm for 72 hours.

During the alcohol fermentation to important sequential pathways, the aerobic pathway and the anaerobic pathway. In the first, the yeast uses the oxygen contained in the must and present in the head space of the digester to asexually reproduce by sprouting. This stage last approximately 12 hours, and once the oxygen is depleted, the conditions become anaerobic and it is here that the yeast begin to produce ethanol. The fermentation conditions affect the conversion rate of the fermentable sugars to bioethanol. Generally, fermentations are carried out with an initial pH of 4.5 at a temperature of between 32 and 36° C. The pH gradually goes down during the fermentation stage due to the production of organic acids and the temperature tends to increase due to the active yeast generates heat that must be dissipated by a heat exchange that generally forms part of the fermentation reactors.

### h) Distill Fermented Mix (8)

For primary recovery of the bioethanol (the principal product of this process), which is mixed with: water and spent distillery grain, the mixture is submitted to a typical distillation process where it is heat treated in a distillation column. In these columns the ethanol evaporates at temperatures above 70° C. and recondenses; generally in the first part of the distillation stage the ethanol is recovered with water (90% ethanol and 10% water). This product is submitted to a second stage of distillation where the ethanol content is rectified to be concentrated at 95%. Finally, to obtain ethanol grade fuel with a purity above 98%, the mixture is submitted to azeotropic distillation or to dehydration using a molecular sieve that binds water to the mixture. The principal product of this stage is the ethanol and the remaining product of these operations is a mixture of the spent distillery grain:water.

### i) Dehydrate Bioethanol in Molecular Sieves (9)

The bioethanol obtained in the distillation stage is dehydrated. For this dehydration several molecular sieves are preferably used packed with zeolite or some similar material. The molecular sieve adsorbs and retained the water present in the bioethanol and anhydrous or dehydrated bioethanol, ready to be used as fuel.

### j) Centrifuge Spent Distillery Grain (10)

The mixture of spent distillery grain:water, obtained in the distillation stage, is centrifuged to separate the spent distillery grain from the water, that may even contain minimal amounts of bioethanol that could have been trapped and reintegrated to the flow that enters into the distillation stages.

The product obtained in this stage is the spent distillery grain, that afterwards may be dehydrated or dried to be channeled into domestic animal feed.

For the purpose of stressing the efficiency of the process which is the motive of this invention, with respect to the conventional process and the conventional process that only incorporates one of the two new stages (decortication or protease hydrolysis), 6 processes were carried out independently, that we will identify as indicated in the following list and in Table No. 2.
(A) Conventional process, for corn (does not include the stages of decortication and protease hydrolysis included in the process here proposed).
(B) Conventional process, for sorghum (does not include the stages of decortication and protease hydrolysis included in the process here proposed).
(C) Conventional process with the incorporation of the decortication stage, for sorghum.
(D) Conventional process with the incorporation of protease hydrolysis, for corn.
(E) Conventional process with the incorporation of protease hydrolysis, for sorghum.
(F) Process here proposed for sorghum, that includes the decortication stage and protease hydrolysis.

TABLE 2
Differences between the conventional process and the
process which is the motive of this invention that
includes decortication and protease hydrolysis.

| | CORN | SORGHUM | | CORN | SORGHUM | |
|---|---|---|---|---|---|---|
| | A | B | C | D | E | F |
| Decortication | No | No | Yes | No | No | Yes |
| Grinding | Yes | Yes | Yes | Yes | Yes | Yes |
| Protease hydrolysis | No | No | No | Yes | Yes | Yes |
| Liquify | Yes | Yes | Yes | Yes | Yes | Yes |
| Saccharification | Yes | Yes | Yes | Yes | Yes | Yes |
| Adjust degrees plato | Yes | Yes | Yes | Yes | Yes | Yes |
| Ferment | Yes | Yes | Yes | Yes | Yes | Yes |
| Distill | Yes | Yes | Yes | Yes | Yes | Yes |
| Dehydrate | Yes | Yes | Yes | Yes | Yes | Yes |
| Centrifuge | Yes | Yes | Yes | Yes | Yes | Yes |

### a) Decorticate the Sorghum Grain

This stage applies to the processes identified as “C” and “F” and consists in taking 3 lots of sorghum weighing 4 kg each, and submitting them to mechanical decortication, using pilot plant equipment fitted with carborundum discs (60 grit) measuring 30 centimeters (Nutana Machine, Saskatoon). These 5 discs are placed on an axis that rotates a high revolutions. When the sorghum grains are submitted to friction on the abrasive surface of the discs, they gradually lose their outer layers. The grain and the fiber removed are through a sieve every 5 minutes to estimate the level of decortication and the yield of by-products, or decorticated material.

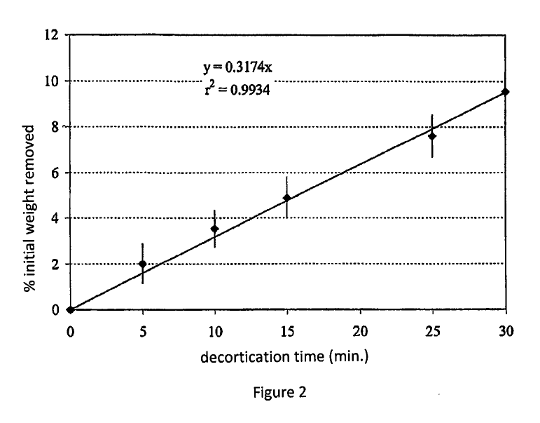

The sorghum grain is submitted to decortication for 30 minutes for the purpose of removing 10% of the original weight of the grain. It should be mentioned that there is other commercial decortication equipment that can perform this work in 5-7 minutes because they operate at greater revolutions per minute, however the decortication is preferably carried out until 10% of the original weight of the grain is removed. In FIG. 2, the equation is recorded that shows the percentage of material removed (Y) or decorticated with respect to the decortication time (X).

### b) Dry Grind the Grain

The dry grinding stage is used separately from all the processes identified in Table 2, as: A, B, C, D, E, and F; preferably using a hammer mill (Arthur Thomas Co. Philadelphia, Pa.) provided with a mesh having 2 mm holes. The resulting sorghum flours contained a particle size distribution or granulometric profile with 68% of the particles measuring 420 μm, 11.5% between 250-420 μm, 7.7% between 149 and 250 μm, and 11.1 lower than 149 μm. Corn flour had a finer particle size distribution or granulometric profile in comparison with the two sorghum flours. Approximately 50% of the particles with a size of 420 μm, 12% between 250-420 μm, 12% between 149 and 250 μm, and 25% smaller than 149 μm. Afterwards, the flours obtained for each one of the 6 independent processes, were described chemically, and the results are shown in Table 3.

TABLE 3
Chemical description and total phenol profile of raw material for the production
of flours of liquefaction and of fiber obtained by decortication of sorghum ¹.

| | | White Sorghum | | Fiber from |
|---|---|---|---|---|
| | | Whole | | Sorghum |
| Parameter | Yellow Corn | Grain | Decorticated | decortication |
| Moisture | 12.03 ± 0.11 | 12.53 ± 0.19 | 11.15 ± 0.05 | 8.88 ± 0.04 |
| Ash | 1.42 ± 0.20 | 1.42 ± 0.08 | 136 ± 0.03 | 3.96 ± 0.05 |
| Protein | 9.26 ± 0.56 | 12.41 ± 0.51 | 10.90 ± 0.62 | 18.07 ± 0.20 |
| Ethereal Extract | 4.00 ± 0.42 | 1.09 ± 0.27 | 0.66 ± 0.05 | 10.43 ± 0.44 |
| Raw Fiber | 3.03 ± 0.18 | 3.65 ± 0.37 | 1.78 ± 0.14 | 11.08 ± 0.24 |
| ELN | 82.28 ± 1.80 | 82.51 ± 1.90 | 83.80 ± 0.84 | 76.26 ± 0.76 |
| Starch | 76.74 ± 0.18 | 73.24 ± 0.24 | 75.80 ± 0.22 | 19.80 ± 0.41 |
| Total Phenols | 0.42 ± 0.01 | 1.44 ± 0.09 | 0.45 ± 0.01 | 10.92 ± 0.72 |
| (equivalent to gallic | | | | |
| acid mg/g) | | | | |

¹ The reported values correspond to the average of three repetitions and are reported as a percentage of the dry base, with the exception of the total phenols.

The 10% decorticated sorghum grain (from the processes identified as: C and F) had lower amounts of ash, ethereal extract, and raw fiber, and this also implies the removal of 68.7% of the total phenol compounds in comparison with processes B and E that employ non-decorticated sorghum grain, and in comparison with the processes A and D, that use non-decorticated corn kernels. This change in the composition produced an increase in the starch content and/or free nitrogen extract content of the decorticated sorghum grain in comparison to the non-decorticated sorghum grain.

Removing phenol compounds also improves the decorticated sorghum grain as a substrate for amylolytic enzymes in the liquefaction and saccharification stages and the yeasts during fermentation.

c) Hydrolyze with Protease

The protease hydrolysis stage the processes identified in Table 2 as: D, E, and F; are separately applied and consists of preparing a 30:70 flour:water suspension (flour obtained in the dry grinding stage), so, for each process 350 mL of water was added to 150 g or dry flour.

To each of the six flour:water suspensions: water is treated separately as described below:
Add 0.2% Ca(OH)₂ in ratio to the flour content.
Adjust the pH to 6.5 using HCl 0.1N.
Add 0.5 mL of a proteolytic enzyme that may optionally be Novo Nordisk's Neutrase® 0.5 L. The supplier states that the activity of this enzyme is 0.5 Anson units/g and that it works optimally at a temperature between 45 and 55° C. and at a pH of between 5.5 and 7.5. Optionally, any other protease or combination of proteases may be added according to the specifications of the suppliers. This protease may be preferably used at the temperature and pH recommended by the supplier.
Heat in a shaking water bath to heat (Hot Shaker, BellCo Glass, Inc. Vineland, N.J. USA) the flour:water suspension, making sure that after 15 minutes a temperature of 60° C. is reached.

In the starch hydrolysis stage, gelatinization occurs, a phenomenon that may be affected by the characteristics of the starch granule.

Once this stage has reached its conclusion and the final reducing sugar concentration obtained in each type of suspension has been determined (corn flour suspension, whole sorghum flour suspension, decorticated sorghum flour suspension), you have:

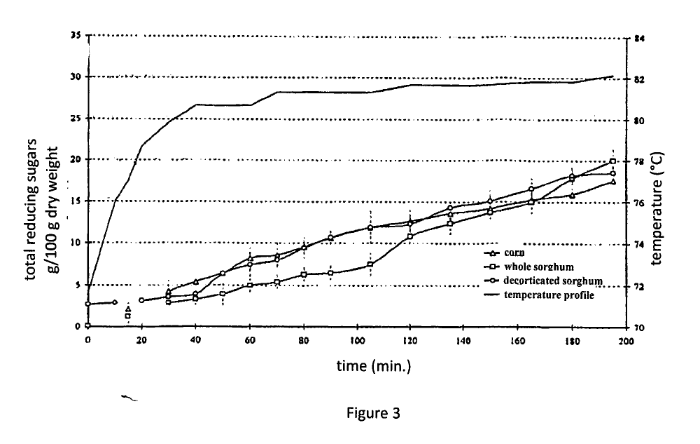

In FIG. 3, it can be seen that in the conventional process (that does not include the protease hydrolysis stage) there are no significant differences in the reducing sugar profile for the three types of grain used, and that the hydrolyzed substances obtained include between 17 and 20% reducing sugars after 3 hours of hydrolysis.

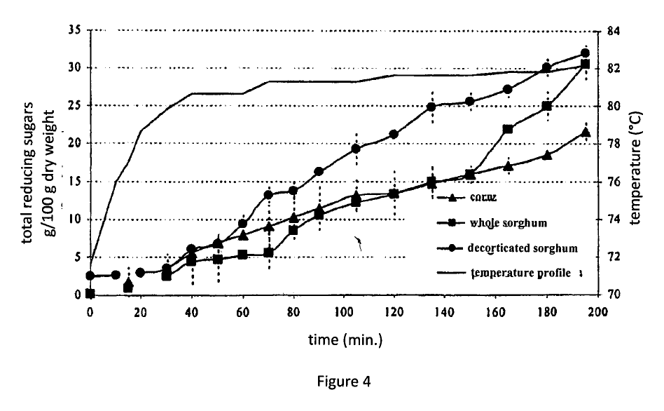

In FIG. 4 it may be seen that by including protease hydrolysis before liquefaction very significantly increased the concentration of reducing sugars, especially in the hydrolyzed substances from sorghums. Comparatively, it is interesting to stress that in the conventional process, the maximum amount of sugars were made up of 15 g of reducing sugars/100 g, while in the sorghums treated with proteases they surpassed 30 g/100 g. This indicates important differences in the rate of hydrolysis and the need to include the protease hydrolysis stage before the liquefaction stage. However, surprisingly, the hydrolyzed substances from corn flour that included the protease hydrolysis stage were those that completed liquefaction with lower quantities of reducing sugars. With the data obtained it may be concluded that including the protease hydrolysis stage before the liquefaction stage produces a greater concentration of reducing sugars, from which bioethanol will be obtained.

### d) Liquefy the Hydrolyzed Flour:Water Suspension

The liquefaction stage, the hydrolyzed flour:water suspension obtained in the protease hydrolysis stage is applied separately from all the processes shown in Table 2, as: A, B, C, D, E, and F; and it consists of preparing:
Add 0.2 mL of heat-resistant alpha-amylase, which may optionally be Termamyl L®, Novo Nordisk, to each of the hydrolyzed flour:water suspensions.
Increase the temperature gradually for 55 minutes until the maximum temperature of 83 to 95° C. is reached.
Allow to react for 2 hours at a temperature of 83° C. in a shaking bath at 150 rpm. Shaking improves hydrolysis since it promotes contact between the alpha-amylase enzyme and the substrate.
Reduce the temperature until it preferably reaches 60° C. as quickly as possible.
Adjust the dextrin suspension to a pH of 5.5 using HCl 0.1 N.
   The product obtained in this stage is called dextrin suspension generated by hydrolysis of the amylase and amylopectin chains.

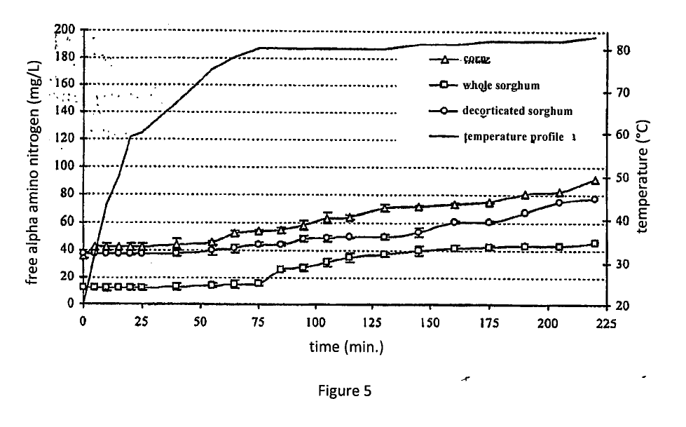

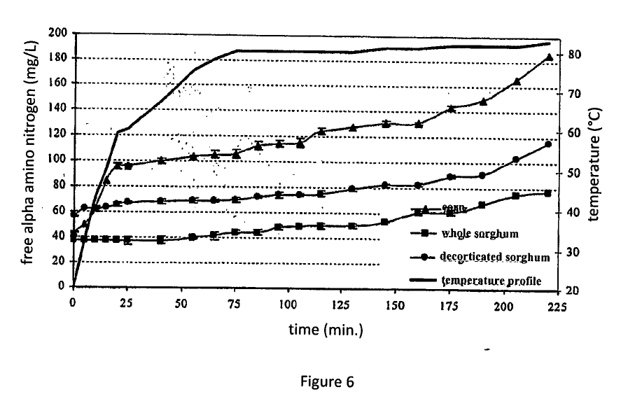

In FIGS. 5 and 6, the free alpha amino nitrogen or α-ANL generated during the liquefaction stage is reported, for the conventional process (FIG. 5) and the process here proposed that includes protease hydrolysis (FIG. 6). In FIG. 5, the α-ANL values obtained from corn, whole sorghum, and decorticated sorghum flours are shown, which have been treated using the conventional process. The corn had the highest values of α-ANL followed by decorticated sorghum and in last place, the whole sorghum. The difference of the α-ANL between whole and decorticated sorghum, is increased in the decorticated sorghum and this is attributed to the decortication stage that removes the pericarp, rich in cellulose, hemicellulose, lignin, and other fibers that act as physical barriers that delay enzymatic hydrolysis and also reduced the concentration of phenol compounds that inhibit enzymes as α-amylase and trypsin, bringing as a consequence lower inhibition of said enzymes. The hydrolyzed substances from decorticated corn and sorghum had approximately 60 and 30% more α-ANL respectively in comparison with the hydrolyzed substances from whole sorghum.

In FIG. 6 the values obtained for α-ANL in corn, whole sorghum, and decorticated sorghum grains treated with the process that is the purpose of this invention are shown, which is evidence that including the protease hydrolysis stage increased the concentration of α-ANL in all types of hydrolyzed substances. This is because the protease partially hydrolyzes the proteins producing greater amounts of low molecular weight amino acids and peptides that are the available nitrogen source for the yeast.

The hydrolyzed substances of whole sorghum and decorticated sorghum that include the protease hydrolysis stage had approximately 40 mg/L more α-ANL as compared with the hydrolyzed substances of whole sorghum and decorticated sorghum obtained by the conventional process.

It is therefore concluded that the integration of mechanical sorghum decortication stages and protease hydrolysis before and during liquefaction is highly advisable because the level of starch and protein hydrolysis is increased significantly during liquefaction, which translates into a substrate that is more apt for amyloglucosidase that is incorporated in the stage after saccharification. A greater concentration of nitrogen is also obtained that eventually will be available for the yeast used in the stage subsequent to fermentation.

### e) Saccharification of the Dextrin Suspension

The saccharification stage of the dextrin suspension obtained in the stage prior to liquefaction, to obtain a concentrated sweet must, is applied separately from the processes identified in Table 2, as D, E, and F; and it consists of:

Adding an amyloglucosidase, preferably Dextrozyme® (Novo Nordisk, Princeton, N.J.) in a ratio of enzyme:substrate 1:100 (p/p). This enzymatic complex mainly consists of amyloglucosidase or glucoamylase and debranching enzymes for the starch chains that are called pullulanases. These enzymes hydrolyze the dextrins resulting from the glucose liquefaction. In the research here described, once the enzymatic complex was added, the temperature was maintained at 55° C. for 16 hours with shaking at 120 rpm. It should be mentioned that this saccharification stage generally includes the fermentation stage in order to have significant savings in terms of the energy and time. When the saccharification stage is integrated into that of the fermentation, the temperature and pH are controlled at between 36 and 37° C., and at 5.2 to 5.4, respectively.

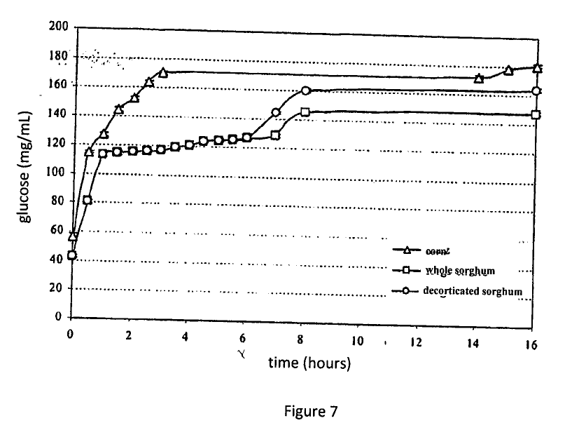

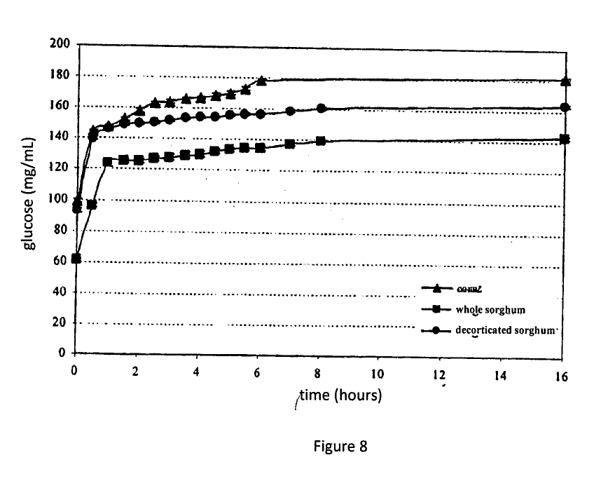

In FIGS. 7 and 8, the glucose concentration (mg/mL) obtained during the saccharification stage is reported, with the conventional process (FIG. 7) and with the process here proposed that included the protease hydrolysis stage (FIG. 8). FIG. 7 is a graph that shows the glucose concentration obtained in the saccharification stage of the conventional process for each of the three different treatments. It can be seen that the glucose concentration obtained in the treatments with whole sorghum and decorticated sorghum is kept practically the same during the first six hours, presenting afterwards a difference in the concentration favoring the former above the treatment with decorticated sorghum. However, in the third hour, the treatment with corn reached the greatest glucose concentration, and there was no significant difference between the concentration observed between the fourteenth and sixteenth hour. However, the final glucose concentration of the three treatments ranged between 140 and 180 mg glucose/mL, with the highest concentration obtained in the treatment using corn flour as the raw material.

It can be seen in FIG. 8 that the glucose concentration is not affected by including the decortication and protease hydrolysis stages, since the glucose concentration range is between 140 and 180 mg/mL, which is the same as in the equivalent treatments in the conventional process; but including said stages in the process here proposed did have an effect on the rate of the conversion of dextrins to glucose, since during the two first hours the difference between the glucose profiles observed in the whole sorghum, decorticated sorghum was minimal; however, at the end of 3 hours, the treatment with corn generated 30% more glucose than that generated by any of the treatments with whole sorghum and decorticated sorghum; and the value reached by the treatment with corn in the first three hours, was not reached by the decorticated sorghum until after eight hours of reaction.

Analyzing the results obtained for the treatments that followed the process which is the motive of this invention, it can be noted that as it was assumed, the whole sorghum presented the lowest hydrolysis rate and the lowest final glucose concentration. The glucose conversion in the treatments with corn and decorticated sorghum were the same during the first 2 hours and afterwards the glucose concentration from the treatment with corn exceeded the glucose concentration from the treatment with decorticated sorghum, so that after 6 hours, the glucose concentration from the treatment with corn exceeded approximately 10% the glucose concentration from the treatment with decorticated sorghum. The final glucose concentration relative to that observed with the hydrolyzed substances of corn was between 88 and 72% for the whole and decorticated sorghum, respectively.

In each of the treatments (corn, whole sorghum, and decorticated sorghum), the use of protease did not affect the final glucose concentration in the hydrolyzed substances, however it did have an effect on the speed at which the glucose was generated. This indicates that the use of protease may reduce biocatalysis time with the amyloglucosidase. In the case of corn, including the protease hydrolysis stage allows the same glucose concentration to be generated in the first 30 minutes as that generated in 90 minutes with the conventional process. Upon integrating the sorghum decortication stage and the protease hydrolysis stage in the process which is the motive of this invention, you have the same concentration of glucose with the decorticated sorghum 30 minutes after starting the saccharification stage as that observed at 7 hours with decorticated sorghum not treated with protease. All this is evidence that the integration in the same process of the decortication stage and the protease hydrolysis stage causes an increase in the speed with which the dextrins are generated during the liquefaction and glucose step during saccharification. This translates into a clear reduction in processing times and provides energy savings.

### f) Adjust to Degrees Plato for the Concentrated Sweet Musts

The stage for adjusting the concentrated sweet must to degrees plato is applied separately from the processes identified in Table 2 as: A, B, C, D, E, and F and it includes:
Cool the concentrated sweet musts obtained by saccharification (Stage E) in preparation for the critical fermentation stage that is generally carried out at a temperature of 25° C.
Measure the concentration of the soluble solids as ° Plato using a refractometer. Only if the concentration of soluble solids in the concentrated sweet musts is above 15° Plato, should you follow with the next step.
Dilute with water until a reading on the refractometer of preferably 13° Plato is reached. The adjustment in ° Plato is necessary to ensure that the glucose concentration does not inhibit the yeast via osmotic pressure. The adjustment may change if yeasts or other osmo-resistant microorganisms are used. There are strains of yeasts that can ferment musts adjusted up to 20° Plato.
   In all the treatments, we began with a glucose concentration that is in terms of the values obtained during the saccharification process (FIGS. 7 and 8) adjusted to the dilution necessary for adjusting the level of fermentable sugars.

### g) Ferment the Standardized Sweet Must

The stage for fermenting the concentrated sweet is applied separately from the processes identified in Table 2 as: A, B, C, D, E, and F and it includes:
Adjust the pH of the sweet musts preferably to 4.5.
Optionally, inoculate the sweet musts with Saccharomyces cereviseae ATCC 24858 in a ratio of 1:100; i.e., the following was added: 1 mL of inoculum per 100 mL of suspension to initiate fermentation with a concentration of 15×106 cells/900 mL of standardized sweet must. For the batch fermentation stage, 1 L capacity Erlenmeyer flasks were used as bioreactors. A maximum and minimum volume of 600 mL and 500 mL were worked with, respectively. All inlets were sealed, leaving only a valve check and the bioreactors were placed in an incubator (Lab-Line Modelo 3526) at 30° C. with shaking at 125 rpm for 72 hours.

It is important to consider that the inoculum Saccharomyces cereviseae ATCC 24858 was previously propagated from a pure culture, and for this, Yeast Maltose Broth (Difco Sparks, Md.) was used as a culture medium. From the original saved culture, 5 mL were taken which were introduced into an OminiPlus® reactor with 2 liters of medium at a temperature of 32° C. and with shaking at 500 rpm at a pH=7.0 for 48 hours, in accordance with the conditions recommended by the ATCC. Once this time has passed, the incubated mixture is removed and centrifuged at 5000 rpm for 5 minutes to collect the precipitate that was stored at −80° C. (REVCO Ultima II Model, Asheville, N.C.) in 5 mL cryopreservation vials, adding 1 mL of glycerol for every 4 mL of precipitate.

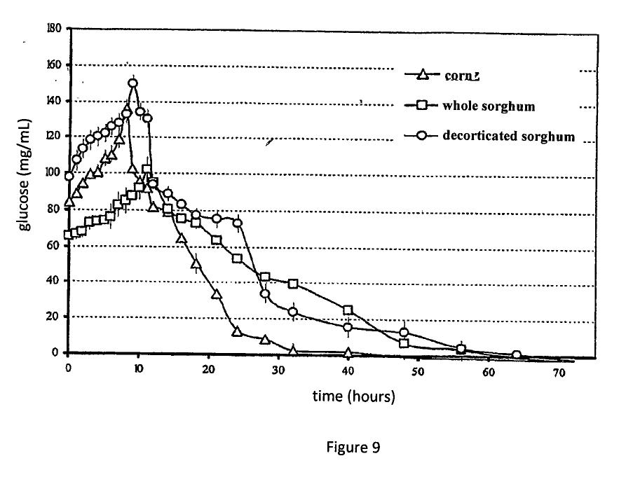

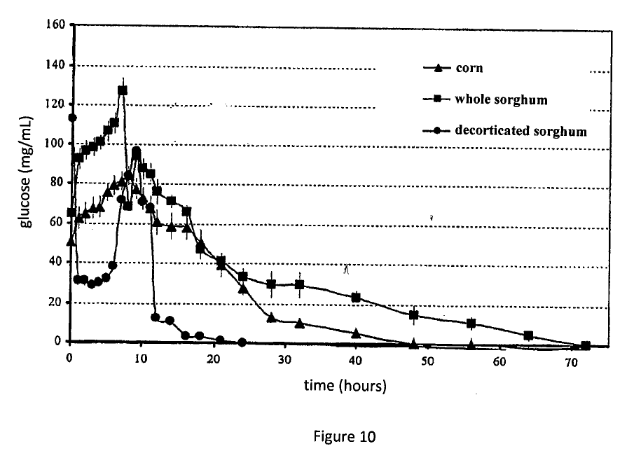

In FIGS. 9 and 10 the profile of glucose consumption may be seen throughout the fermentation stage with Saccharomyces cereviseae ATCC 24858 for each of the six different treatments.

During the fermentation stage a process to generate and consume glucose takes place. It begins with a glucose concentration that is in terms of the values obtained during the saccharification stage (FIGS. 7 and 8) adjusted to the dilution necessary to adjust the level of fermentable sugars. This initial concentration increases until a maximum value is obtained, for a gradual reduction afterwards until reaching a value of “zero” (indication that the fermentation and production of ethanol was carried out completely).

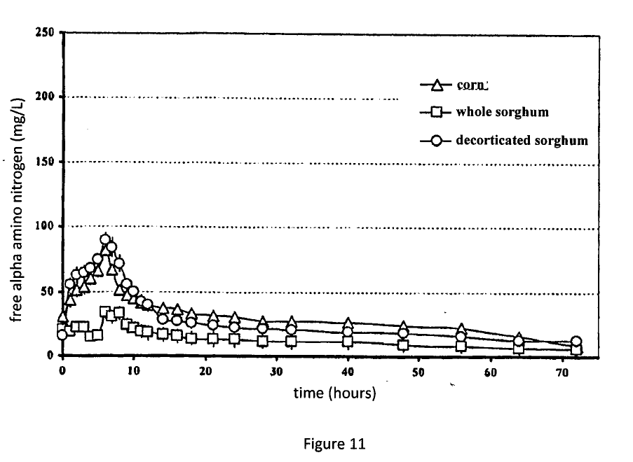

In FIG. 11 the behavior of the α-ANL concentration is plotted at different times during fermentation. The maximum value of α-ANL was reached at 7 hours and after 14 hours of fermentation there are no significant differences for the three types of musts.

Among the maximum concentrations of α-ANL reached by each treatment, we have the maximum corn concentration is approximately 220 mg/L, it is approximately 150 mg/L in decorticated sorghum, and it is less than 50 mg/L in whole sorghum, in a period of approximately 7 hours. As the time passed, it may also be observed that in all of the cases, the values reduced in such a way that at 8 hours there was no significant difference between the concentration of α-ANL in corn and decorticated sorghum. When the α-ANL concentration obtained in each of the treatments submitted to the conventional process is compared with similar treatments using the process which is the motive of this invention, it was observed that the musts obtained with the process which is the motive of this invention reached values of α-ANL concentration were three times greater in comparison with the musts obtained with the treatments submitted to the conventional process.

In the conventional process, there was no significant difference (p>0.05) between treatments with corn and decorticated sorghum after 21 hours of fermentation. But the treatments with corn and decorticated sorghum submitted to the process which is the motive of this invention, reached almost double the concentration of α-ANL in comparison with the musts from the treatments submitted to the conventional process.

After 40 hours of fermentation, the α-ANL concentration went down in both processes (conventional and that here proposed) and the difference between the six treatments was not significant (p>0.05).

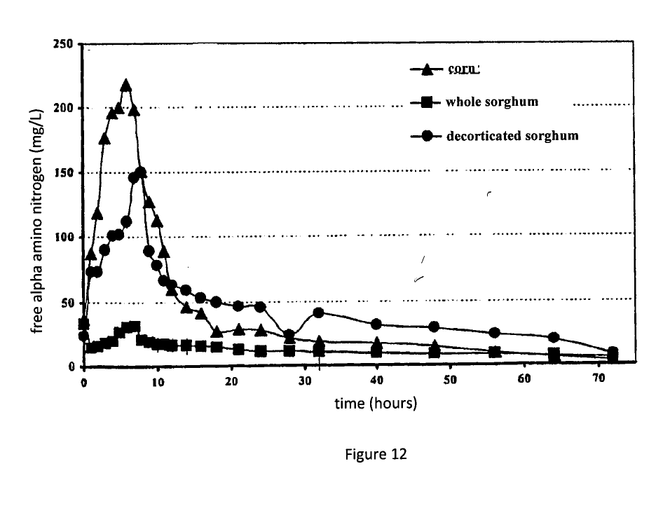

In the α-ANL concentrations for the treatment with sorghum, submitted to the conventional process and the process which is the motive of this invention, no significant differences existed. (FIGS. 11 and 12).

The highest value of α-ANL was present in the musts obtained from the treatment with corn submitted to the process which is the motive of this invention and the null effect of this treatment on the profile of the reducing sugars during the liquefaction and that of the glucose during saccharification and fermentation, confirming the occurrence of a lower level of interaction between the protein matrix and the corn starch.

In the treatment with whole sorghum submitted to the process which is the motive of this invention, implementing the protease hydrolysis stage affected the α-ANL concentration probably because the fiber contains enzymatic inhibiting components such as phenols or simply through the impediment of the rapid enzymatic attack due to the presence of the fibrous pericarp itself.

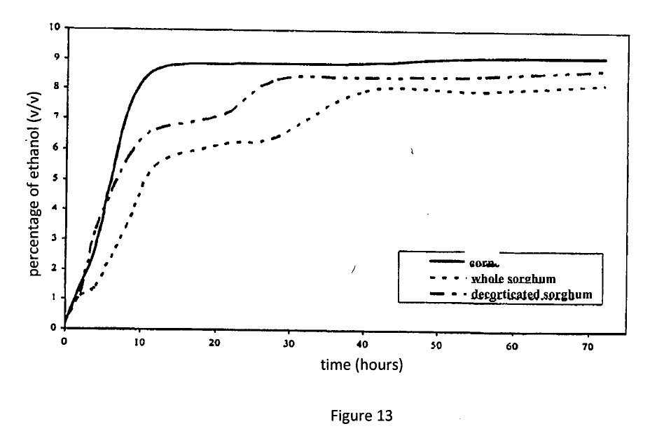

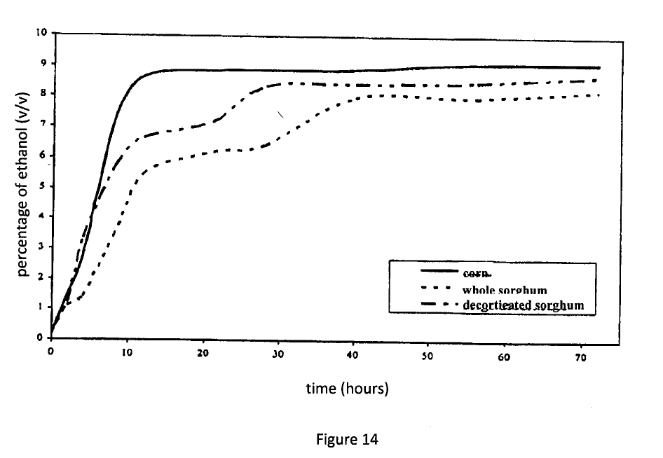

FIGS. 13 and 14 present profiles of obtaining bioethanol during fermentation of the musts from different treatments submitted to the conventional process and the process which is the motive of this invention.

In FIGS. 13 and 14 it may be seen that the final maximum concentration of bioethanol was obtained with corn submitted to both processes (conventional and that here proposed). The generation of bioethanol from corn, through time showed a behavior that was very similar when both processes were compared. On the other hand, including only the decortication stage of sorghum, made it possible to obtain three times more ethanol concentration in 4 hours as compared with the counterpart produced from whole sorghum (FIG. 13).

For treatments with whole sorghum and decorticated sorghum, including the protease hydrolysis stage increased the rate of generation of bioethanol (FIG. 14).

By analyzing the effect of only integrating the protease hydrolysis stage, it was observed that after 12 hours of fermentation the bioethanol concentration was 1.5 times more with whole sorghum with protease in comparison with whole sorghum without protease and the final bioethanol concentration was 5% higher using decorticated sorghum treated with protease than without protease. After 12 hours of the fermentation stage, the bioethanol concentration generated in the musts of the treatment with decorticated sorghum submitted to the process which is the motive of this invention, was 1.8 times greater than that observed in the must from the treatment with decorticated sorghum submitted to the conventional process (that does not include the protease hydrolysis stage) The final bioethanol concentration was 2.6% more for decorticated sorghum submitted to the new process that includes protease hydrolysis than for the decorticated sorghum submitted to the conventional process that does not include the protease hydrolysis stage.

With respect to sorghum (FIG. 14), submitted to the process here proposed that includes the decortication and protease hydrolysis stages produced a change in the bioethanol generation profile, making it possible to obtain concentrations that are closer to those observed in corn, but in less time.

Just including the protease hydrolysis stage in whole sorghum also increased the ethanol concentration but this was achieved in the last stages of fermentation.

In the case of including the protease hydrolysis stage with decorticated sorghum, it presents significant bioethanol concentrations. It is important to note that in the whole sorghum musts the glucose is also depleted, even though much later than for the other grains.

With sorghum, the greatest yield in bioethanol was obtained when the grain decortication stages and protease hydrolysis were included before and during the first part of the liquefaction stage while the lowest ethanol yield was present with the whole sorghum processed by the conventional process (see TABLE 4).

TABLE 4
Balance of material in the saccharification and fermentation stage and bioethanol
yield values in terms of flour and starch that entered into the system.

| | | Starch | Residual | Glucose | | | Ethanol Yield | |
|---|---|---|---|---|---|---|---|---|
| | Flour¹ | Flour | Starch | Output | Ethanol | L/kg | (L/kg Flour) | |
| Raw Material | (g) | (g) | (g) | (g) | (mL) | Starch | Dry Base | Wet Base (14%) |
| | Corn | | | | | | | |
| Without Protease | 100 | 76.74 | 1.51^{c, d} | 64.83^{a, b} | 44.17^{c} | 0.576^{c} | 0.442^{c} | 0.380^{c} |
| With Protease | 100 | 76.74 | 1.31^{d} | 65.43^{a} | 45.67^{b} | 0.595^{b} | 0.457^{b} | 0.393^{b} |
| | Whole Sorghum | | | | | | | |
| Without Protease | 100 | 73.24 | 2.82^{a} | 50.42^{d} | 32.40^{c} | 0.442^{e} | 0.324^{e} | 0.279^{e} |
| With Protease | 100 | 73.24 | 2.13^{b} | 61.05^{c} | 42.43^{d} | 0.579^{d} | 0.424^{d} | 0.365^{d} |
| | Decorticated Sorghum | | | | | | | |
| Without Protease | 100 | 75.80 | 1.54^{c} | 60.84^{c} | 45.12^{b} | 0.595^{b} | 0.451^{b} | 0.388^{b} |
| With Protease | 100 | 75.80 | 1.30^{d} | 63.98^{b} | 46.71^{a} | 0.616^{a} | 0.467^{a} | 0.402^{a} |

¹The weight of flour when entering into the system is expressed as a dry base

The integration of decortication and the use of protease increased the ethanol yield produced from sorghum by 44%. If the ethanol yield from decorticated sorghum treated with protease is compared with corn treated with protease, there was no significant difference. This indicates that it is possible to equal the bioethanol yields to those commonly obtained with corn if the innovative process here proposed is used. The amounts recovered from spent grain or GGD for each treatment is summarized in TABLE 5.

TABLE 5
Yield from spent grain after the fermentation of
corn, and whole and decorticated sorghum musts in terms
of dry weight of the original grain¹.

| Raw Material | Percentage |
|---|---|
| Yellow Corn | |
| Without Protease | 30.89^{b} |
| With Protease | 29.41^{b} |
| Whole Sorghum | |
| Without Protease | 35.20^{a} |
| With Protease | 30.17^{b} |
| Decorticated Sorghum | |
| Without Protease | 30.18^{b} |
| With Protease | 27.20^{b} |

¹Each value is expressed in wet base and is the average of three repetitions. The different letters in the same column represent statistically different values.

Just as was expected, the whole sorghum processed under the conventional scheme produced the greatest percentage of GGD. This is because it was not decorticated, its protein was more difficult to hydrolyze, and probably because the fiber and protein trapped some starch granules that did not gelatinize nor hydrolyze with the heat-stable α-amylase. The use of protease reduces the amount of spent grain by 5% (TABLE 4). With respect to the chemical composition of these GGD (TABLE 4) in all cases, the moisture values were high, which basically causes the by-product to be very perishable. The proximal chemical composition of the GGD indicates little difference when corn and whole sorghum were compared. However, the decorticated sorghum spent grain had a lower fiber and ethereal extract content since during decortication the pericarp and germ were selectively removed. The high content of protein observed in all spent grain indicates that these by-products may be valuable in animal feed, principally for ruminants. The spent grain obtained from decorticated sorghum may possible be used in monogastric animal feed due to its lower fiber content. The protein concentration was greater in the whole sorghum GGD as well as with or without protease and lower values were observed in the corn with or without protease and decorticated sorghum with protease. The ethereal extract concentration was greater in the case of the corn GGD without protease and the lowest was from whole sorghum without protease and decorticated with or without protease. In regards to the content of raw fiber, the highest values were observed in whole sorghum without protease and the lowest values in decorticated sorghum with or without protease.

In the case of whole sorghum without protease the higher protein concentration was preserved (TABLE 6) and this agrees with that observed in the production of α-ANL during the protease and liquefaction treatment (FIG. 5) while with whole sorghum without protease shown the lower concentration. These spent grains also shown the greatest concentration of residual starch reaffirming the theory that the fiber and protein trapped the starch granules. The lowest concentrations of starch were shown in the yellow corn spent grain and in the decorticated sorghum treated with protease. When analyzing the percentage of raw fiber in the spent grain, it may be seen that in the majority of the cases between 80 and 98% were preserved with the exception of the whole sorghum protease treatment that only retain 66.9% of the raw fiber in comparison with the original. In this case, the losses of raw fiber is attributed to the filtration system, since a mesh with a 3 mm grid on the side was used, which allowed the most fine fiber to be lost.

TABLE 6
Residual composition of the spent grain in terms
of the presence of the compounds in the original flours.
Recovery values expressed based on dry¹.

| | | | | Raw | | |
|---|---|---|---|---|---|---|
| | Ash | Protein | Ethereal | Fiber | ELN | Starch |
| Treatment | % | % | Extract % | % | % | % |
| Yellow Corn | | | | | | |
| Without | 24.80 | 84.31 | 49.21 | 80.82 | 20.60 | 1.97 |
| Protease | | | | | | |
| With Protease | 16.10 | 82.02 | 26.87 | 81.86 | 23.53 | 1.71 |
| Whole Sorghum | | | | | | |
| Without | 29.58 | 99.46 | 77.61 | 92.87 | 22.56 | 3.85 |
| Protease | | | | | | |
| With Protease | 54.70 | 84.13 | 89.13 | 66.87 | 18.96 | 2.91 |
| Decorticated | | | | | | |
| Sorghum | | | | | | |
| Without | 22.56 | 81.59 | 85.66 | 97.66 | 22.36 | 2.03 |
| Protease | | | | | | |
| With Protease | 24.04 | 65.79 | 92.73 | 82.36 | 21.03 | 1.71 |

¹Each value is the average of three repetitions.

Especially with the decorticated sorghum treated with protease, the bioethanol yields obtained set the guideline to present a process that makes yields equal to those obtained with corn possible and greater by 40% in the case of whole sorghum without protease treatment. The flow chart for this process is found in FIG. 1. Generally speaking, the process is divided into 9 stages, and the stages that are added to the conventional process are: mechanical decortication before dry grinding and protease hydrolysis prior to and during the first part of liquefaction. Some aspects to keep in mind is being careful with the fiber separation system during decortication in order to avoid the loss of starch in the cracked grain and the size of the particle obtained during grinding, controlling the temperature and pH during the liquefaction, saccharification, and fermentation stages. In the latter, special care must be taken to avoid contamination with microorganisms that can consume the substrates which would reduce the ethanol concentration in the final product. One of the advantages of this process is to obtain a storage-stable decortication fiber suitable for ruminant feed, the intake of more starch per kilogram of flour in the process and a clear and significant reduction in the liquefaction, saccharification, and fermentation times. These savings in time will surely translate into savings in energy, labor, and in a more efficient use of the ethanol biorefinery. It is possible that the decorticated sorghum spent grain may be able to be channeled to monogastric animal feed due to its high protein content and low fiber content.

### h) Distill Fermented Mix

The distilling stage for the fermented mixture obtained in the previous stage, is applied separately from the processes identified in table 2, as: A, B, C, D, E, and F and consists of heating preferably to 80° C. The mixture contains alcohol, water, and spent distillery grain. Bioethanol reaches its boiling point at approximately 70° C. and enters into a distillation column, and is condensed as a bioethanol:water mixture, with a ratio of 95:5 respectively; and the residual that is left is a mixture of spent distillery grain:water.

### i) Dehydrate Bioethanol

The dehydration stage of the bioethanol is applied separately from the processes identified in table 2, as: A, B, C, D, E, and F and consists of dehydrating the bioethanol obtained in the distillation using molecular sieves packed with zeolite or any similar material, to obtain bioethanol ready to be used as fuel.

### j) Centrifuge Spent Distillery Grain

The centrifuge stage for the residual spent distillery grain in the mixture that entered into the distillation stage and it is applied separately from the processes identified in table 2 as: A, B, C, D, E, and F it consists in:

Centrifuge the spent distillery grain at 3000 rpm for 20 minutes to extract the water:ethanol that may be trapped and to reintegrate it to the flow that enters into distillation. The spent distillery grain is the precipitate obtained.

Having sufficiently described our invention, we feel that it is an innovation and therefore, claim the content of the following clauses as our exclusive property:

1. Improved process to obtain bioethanol from sorghum grain (Sorghum bicolor L. Moench) characterized in that it includes the stages of:
a) Decorticate the sorghum grain until between 7 and 10% of the original weight of the grain is removed,
b) Dry grind the decorticated grain to obtain a flour with a particle size that permits greater efficiency in the enzymatic hydrolysis and fermentation stages. Generally speaking, the flours must have a granulometric profile of at least 68% of the particles having a size of 420 μm, 11.5% between 250 and 420 μm, 7.7% between 149 and 250 μm, and 11.1 lower than 149 μm.
c) Hydrolyze a flour:water suspension with at least one protease,
d) Liquefy the suspension obtained in c) with an alpha-amylase that is preferably heat-resistant,
e) Saccharify the dextrin suspension obtained in d) with amyloglucosidase or an amyloglucosidase/pullulanases complex,
f) Adjust the concentration of the concentrated sweet must between 11-15° Plato,
g) Ferment the sweet must with yeast obtained in f),
h) Distill the fermented mixture,
i) Dehydrate bioethanol,
j) Centrifuge spent distillery grain.

2. Improved process to obtain bioethanol from sorghum grain (Sorghum bicolor L. Moench) in accordance with claim 1, characterized in that stage a) decortication of the sorghum grain is mechanical.

3. Improved process to obtain bioethanol from sorghum grain (Sorghum bicolor L. Moench) in accordance with claim 1, characterized in that stage a) decortication of sorghum grain is carried out preferably in a range of between 7 and 10% of weight to minimize losses of endosperm which contains the starch.

4. Improved process to obtain bioethanol from sorghum grain (Sorghum bicolor L. Moench) in accordance with claim 1, characterized in that stage c) enzymatic hydrolysis is performed with at least one protease.

5. Improved process to obtain bioethanol from sorghum grain (Sorghum bicolor L. Moench) in accordance with claim 1, characterized in that stage c) enzymatic hydrolysis is performed with proteases, is carried out before liquefaction.

6. Improved process to obtain bioethanol from sorghum grain (Sorghum bicolor L. Moench) in accordance with claim 1, characterized in that stage c) enzymatic hydrolysis is performed at a pH of 6.5 and at a temperature of 60° C. or well integrated in the fermentation process that is carried out at a pH of 4.5 to 6.5 and at a temperature of between 32 and 36° C.

7. Improved process to obtain bioethanol from sorghum grain (Sorghum bicolor L. Moench) in accordance with claim 1, characterized in that stage g) fermentation is performed preferably with Saccharomyces cereviseae ATCC 24858 in a ratio of 1:100.

8. Spent distillery grain obtained in accordance with the process of claim 1, characterized in that the average moisture value is 72.75%, ash is 1.08%, protein is 26.36%, ethereal extract is 2.25%, raw fiber is 5.39%, free nitrogen extract or ELN is 64.78%, and starch is 4.75%.
